(19) United States
(12) Patent Application Publication
(10) Pub. No.: US 2004/0083505 A1
(43) Pub. Date: Apr. 29, 2004

(54) Gene expression from the internal ribosome entry site (ires) of rhopalosiphum padi virus (rhpv)

(76) Inventors: Lisa Oriel Roberts, Guildford (GB); Graham John Belsham, Guildford (GB)

Correspondence Address: Greenlee Winner and Sullivan, Suite 201, 5370 Manhattan Circle, Boulder, CO, 80303, US

(21) Appl. No.: 10/344,501

(22) PCT Filed: Aug. 10, 2001
(86) PCT No.: PCT/GB01/03609

(30) Foreign Application Priority Data
Aug. 10, 2000 (GB) 0019745.9

(51) Int. Cl. A01H 1/00; C12N 15/82

(52) U.S. Cl. 800/288; 435/69.1; 435/468; 435/320.1

(57) ABSTRACT

Polynucleotide sequence capable of directing translation initiation in a plant expression system in a cap-independent manner, wherein the polynucleotide sequence directs translation initiation from an AUG codon. In particular, the invention relates to a polynucleotide sequence which comprises the 5′ UTR of Rhopalosiphum padi virus (RhPV) which comprises an internal ribosome enty site (IRES).

The present invention relates to polynucleotide sequences with an effect on gene expression, in particular to those with an effect on translation of mRNA.

More specifically the invention relates to polynucleotide sequences derived from picornaviruses and picorna-like viruses.

Picornaviruses have a single stranded, positive sense RNA genome of about 8 kb. This RNA functions as mRNA in the host, and contains a single large open reading frame (ORF) encoding a polyprotein, which is processed into the mature viral proteins. Translation of picornavirus RNA is dependent on the presence of an Internal Ribosome Entry Site (IRES) within the 5′ untranslated region (UTR). During infection with many picornaviruses, host cell protein synthesis is inhibited following the cleavage of translation initiation factor 4G (eIF4G), but picornavirus translation continues, as it uses a cap-independent mechanism. This phenomenon was first demonstrated by Pelletier & Sonenberg in 1988 [Pelletier, J and Sonenberg, N. (1988). Nature 334: 320-325] using artificial bicistronic mRNA's.

Rhopalosiphum padi virus (RhPV) is an insect virus which infects a narrow range of aphid species of the Rhopalosiphum and Schizaphis families. Based on sequence data, it is considered to be a member of a group of insect picorna-like viruses. Other members of the group include Drosophila C virus (DCV), Plauti stali intestinal virus (PSIV) and Cricket paralysis virus (CrPV). The nucleotide sequence data suggests that the positive sense RNA genome of RhPV encodes two polyproteins from two large ORF's. ORF1 encodes the non-structural proteins which possess sequence similarity to mammalian picornaviruses and plant comoviruses. ORF2 encodes the three structural proteins, which also show similarity to picornavirus proteins. Both ORF's are preceded by long UTR's (of about 500-800 nt). The 5′ UTR of RhPV is predicted to be 580 nt in length, with initiation of protein synthesis believed to occur at the third AUG [Moon et al., 1998, Virology 243:54-65]. This 5′ UTR is highly A/U rich and is predicted to form extensive secondary structure.

The intergenic regions (termed ‘IGR’s) of RhPV and PSIV have recently been shown to contain IRES elements. Moreover, both the IGR and the 5′ UTR of CrPV have very recently been shown to contain IRES elements [Wilson, J. E et al., (2000). Mol. Cell Biol. 20, 4990-4999]. Both the 5′ UTR and IGR IRES elements were reported to function in insect cells and within the rabbit reticulocyte (RRL) in vitro translation system.

Until very recently, no known IRES element had been shown to function in the wheat germ TNT system, i.e. a plant expression system. One IRES has now been shown to function in such a system, the intergenic IRES of CrPV (unlike the 5′ UTR IRES of CrPV which does not). Initiation of CrPV ORF2 from this element takes place at a CCU codon (Wilson, J. E et al., (2000). Mol. Cell Biol. 20, 4990-4999), rather than AUG, so that translation commences with a proline residue.

Genetic elements for use in translation initiation in plants are limited, and there is a need for additional elements (such as IRES sequences) which function efficiently in plants and plant expression systems to modulate translation.

The present invention provides such additional elements which modulate translation in plant systems.

In a first aspect the present invention provides a polynucleotide sequence capable of directing translation initiation in plant expression systems in a cap-independent manner, wherein the polynucleotide sequence directs translation initiation from an AUG codon.

In an alternative aspect, the present invention provides a polynucleotide sequence capable of directing translation initiation in plant expression systems in a cap-independent manner, wherein the polynucleotide sequence directs translation initiation from an AUG or ATG codon, and has at least 10% of the initiation activity of the 5′ UTR of RhPV.

Specifically the polynucleotide sequence of the invention is capable of directing translation initiation in a plant expression system in the presence or absence of a cap translation mechanism.

In particular, the invention relates to a polynucleotide sequence as defined above which comprises the 5′ UTR of RHPV sufficient to direct initiation. The present invention particularly provides a functional equivalent of the 5′ UTR of RPV having at least 10% of the initiation activity thereof. It is preferred that the sequence comprises the 5′ UTR of RhPV (as defined in SEQ ID NO. 1), or a variant thereof.

It will be appreciated that plant expression systems not only relate to in vitro systems, such as cells and cell extracts, but also relate to whole plants, as appropriate.

The present invention provides significant advantages over the prior art. The only known IRES sequence which functions in the wheat germ TNT system (the IGR IRES of CrPV) initiates from a CCU codon. This CCU triplet encodes proline. Accordingly, any potential protein of interest expressed from this IRES would have an extra (foreign) amino acid on the N-terminus. The addition of such an additional proline residue may influence protein structure and function. In contrast, in the present invention, we have identified an IRES sequence derived from RHPV 5′ UTR (Seq ID NO 1) which initiates translation at an AUG codon. This triplet encodes methionine, which is the naturally occurring first amino acid for the vast majority of proteins. Accordingly, using the present invention, the RhPV 5′ UTR can be fused directly upstream of any protein sequence which begins with a methionine to express this protein in a plant or plant expression system, without adversely affecting protein structure and function by the addition of additional amino acid residues (such as proline) at the protein N-terminal end.

The polynucleotide sequence of the present invention is preferably a DNA sequence, more preferably a double stranded DNA sequence. When the nucleotide sequence of the present invention is DNA, the DNA codon at which point translation will be initiated is ATG, although it will be appreciated that translation occurs at the RNA level, and will therefore begin at the equivalent ‘AUG’ codon in the RNA corresponding to any such DNA sequence. Alternatively, the polynucleotide sequence of the present invention may be RNA, in which case the codon at which translation is initiated is AUG.

Preferably, the polynucleotide sequence of the present invention is, or comprises, an IRES element, more preferably being derived from an insect virus, most preferably derived from RHPV. Still more preferably, the polynucleotide sequence of the present invention is derived from a 5′ UTR region, preferably immediately preceding ORF1 in RhPV.

Suitably, the polynucleotide sequence consists of 579 nucleotides immediately preceding ORF1 in RhPV. This polynucleotide sequence is shown as nucleotides 1- 579 of SEQ ID NO: 1. However, the invention is not limited to this full sequence, and extends to cover both 5′ and 3′ deletions of this sequence which do not significantly affect the activity of the polynucleotide sequence in translation initiation, as well as variants thereof.

The present invention extends to variants of the RhPV 5′ UTR initiation sequence. A “variant”, as the term is used herein, includes truncated versions of the reference sequence, and refers to a polynucleotide that differs from the reference polynucleotide, such as by mutation and/or deletion, but which retains essential properties thereof. In the present invention, the essential property is at least 10% of the initiation activity of the RhPV 5′ UTR initiation sequence. A typical variant of a polynucleotide differs in nucleotide sequence from the reference polynucleotide. Thus, a variant reference polynucleotide may differ in base sequence by one or more substitutions, additions, or deletions in any combination for example, in accordance with the natural degeneracy associated with the genetic code. A variant of a polynucleotide may be a naturally occurring such as an allelic variant, or it may be a variant that is not known to occur naturally. Non-naturally occurring variants of polynucleotides may be made by mutagenesis techniques or by direct synthesis.

Within the variants we include altered polynucleotides. “Altered” nucleic acid sequences include those sequences with deletions, insertions, or substitutions of different nucleotides. Included within this definition are polymorphisms which may or may not be readily detectable using any particular oligonucleotide probe, and improper or unexpected hybridisation to allelic variants, with a locus other than the normal chromosomal locus for the polynucleotide sequence encoding the RhPV 5′ UTR initiation sequence.

For example, the present invention extends to 5′ deletions of 100 nucleotides, or more, and 3′ terminal deletions of 200 nucleotides or more of this 5′ UTR sequence of RhPV. We have demonstrated translation initiation activity for such constructs. Indeed, any suitable truncations of the polynucleotide sequence of the present invention are suitably provided such that they allow translation initiation at an appropriate level. We prefer that this level is at least 5%, preferably 10%, more preferably 25% of the activity which may be obtained using the full length 579 base pair 5′ UTR derived from RhPV. More preferably, the polynucleotide sequence invention has at least 40% of this activity, more preferably 60%, or even higher activity relative to the full length 579 base pair 5′UTR derived from RHPV. Appropriate levels of activity for any given use may be readily determined by the person skilled in the art and deletions from either the 5′ end, 3′ end, or both, used accordingly. Appropriate techniques to make such deletions are well known in the art [see, for example, Sambrook, J., et al., (1989) 2nd edition, Cold Spring Harbor Laboratory press—the teachings of this and all subsequent citations referred to herein being hereby incorporated by reference].

The present invention also extends to polynucleotide sequences which have one or more nucleotides deleted, inserted or substituted, provided that the polynucleotide sequence has sufficient activity in initiation of translation, as defined above. The present invention also extends to polynucleotide sequences capable of hybridising to the polynucleotide sequence of the present invention, and which also retain appropriate functional activity. Hybridisation may be assessed using standard techniques under conditions of 6×SSC, 60° C. or 68° C., optionally with 50% formamide (Sambrook et al, supra).

Variants of the initiators of the present invention include hybridising polynucleotides. In particular, the invention extends to such variants which are polynucleotides capable of hybridising with the antisense polynucleotide of SEQ ID NO. 1. Such hybridising polynucleotides are subject to all of the same considerations as other initiators of the present invention, such as length and efficacy, by contrast with the naturally occurring 5′ UTR of RhPV initiation sequence.

A hybridising polynucleotide preferably hybridises under stringent conditions to the target polynucleotide, the stringency conditions being selected to reflect a degree of substantial identity as discussed herein. For example, the conditions preferably give hybridisation when there is about 80% identity or more, such as from 85% identity, from 90% identity or from 95% identity or more.

More specifically, stringency, as it is commonly used in the art, is defined in Wahl, G. M. and S. L. Berger (1987; Methods Enzymol. 152:399-407) and Kimmel, A. R. (1987; Methods Enzymol. 152:507-511). For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridisation can be obtained in the absence of organic solvent, e.g., formamide, while high stringency hybridisation can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridisation time, the concentration of detergent, e.g., sodium dodecyl sulphate (SDS), and the inclusion or exclusion of carrier DNA, are well known to those skilled in the art. Various levels of stringency are accomplished by combining these various conditions as needed. In a preferred embodiment, hybridisation will occur at 30° C. in 750 mM NaCl, 75 mM trisodium citrate, and 1% SDS. In a more preferred embodiment, hybridisation will occur at 37° C. in 500 mM NaCl, 50 mM trisodium citrate, 1% SDS, 35% formamide, and 100 μg/ml denatured salmon sperm DNA (ssDNA). In a most preferred embodiment, hybridisation will occur at 42° C. in 250 mM NaCl, 25 mM trisodium citrate, 1% SDS, 50 % formamide, and 200 μg/ml ssDNA. Useful variations on these conditions will be readily apparent to those skilled in the art.

The washing steps which follow hybridisation can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

The present invention also extends to sequences having sequence identity to the full length 579 nucleotide 5′UTR derived from RhPV, for example 50%, 70%, 80%, 90%, 95% or higher identity (as assessed by the GCG package, University of Wisconsin), and which retain the function of cap-independent translational initiation in plants or plant extract coupled transcription and translation systems. Preferably sequences having such sequence identity also form a similar or identical secondary structure to that of the 579 base pair 5′UTR derived from RhPV, such that the sequences have the same function as translational initiators.

The 5′UTR region of picornaviruses is thought to contain a high degree of secondary structure, generally caused by complementary polynucleotide sequences hybridising to one another. Accordingly; the present invention also extends to sequences which form a similar or identical secondary structure to the 579 nucleotide 5′UTR derived from RhPV, as assessed by the Mfold program (GCGpackage) or other suitable modelling system. This allows the sequence to accommodate nucleotide changes at one first site which are then compensated by changes at a distal site, with which the first site is involved in secondary structure formation, to ensure a similar secondary structure and function.

‘Capping’ of mRNA at the 5′ end generally occurs to allow attachment of the mRNA to the ribosome. The general mechanism is now well understood (see, for example, Stryer, ‘Biochemistry’, 1988). However, the polynucleotide sequence of the present invention functions in a cap-independent manner. In particular, we prefer that the polynucleotide sequence of the present invention may be able to direct translation and protein synthesis even after cleavage of translation initiation factor 4G(eIF4G).

The requirement for the polynucleotide sequence of the present invention to be capable of initiating translation in a cap-independent manner generally refers to the ability of the polynucleotide sequence to act as an internal ribosome entry site (IRES). Essentially, the polynucleotide of the invention directs translation of the RNA into protein to begin at the defined point at the 3′ end of the sequence, to initiate from an AUG codon in the RNA to produce a methionine residue at the front of any given ORF.

The present invention also extends to vectors comprising the polynucleotide sequence of the present invention, preferably operably linked to a gene or open reading flame, such that the polynucleotide sequence allows translation of that gene or open reading frame to be initiated. More preferably, the polynucleotide sequence of the present invention is genetically fused immediately prior to the gene or open reading frame, such that translational initiation occurs at the first AUG (methionine) codon. Vectors may also additionally comprise a selectable marker, in order to enable transformation events into suitable cell lines to be selected and identified.

The invention also extends to cells containing the vectors of the present invention, and to expression systems containing cells of the present invention, which may be cultured in such a way that desired proteins are produced under translation control of the polynucleotide sequence of the present invention. Any suitable cell or expression system may be used in the present invention, and suitable examples of such cells and systems are well known in the art. Both prokaryotic and eukaryotic cells may be used, although we prefer that eukaryotic cells, such as plant cells, are used.

The invention also relates to plants which have been transformed with vectors of the present invention, including transgenic plants, in which certain proteins may be translated in a cap-independent manner. Methods to transform plants, and methods of producing transgenic plants, are well known in the art.

DNA can be transformed into plant cells using any suitable technology, such as: a disarmed Ti-plasmid vector carried by Agrobacterium, exploiting its natural gene transfer ability [EP-A-270355, EP-A-0116718,NAR 12(22) 8711 -87215 1984]; particle or microprojectile bombardment (U.S. Pat. No. 5,100,792, EP-A-444882, EP-A434616); microinjection [WO 92/09696, WO 94/00583, EP-A-331083, EP-A-175966, Green et al., (1987) Plant Tissue and Cell Culture, Academic Press]; electroporation (EP-A-290395, WO 8706614); other forms of direct DNA uptake (DE-A-4005152, WO 9012096, U.S. Pat. No. 4,684,611); liposome mediated DNA uptake [e.g. Freeman et al., Plant Cell Physiol. 29: 1353 (1984)]; or the vortexing method [e.g. Kindle, PNAS US.A. 87: 1228 (1990d)]. Physical methods for the transformation of plant cells are reviewed in Oard, 1991, Biotech. Adv. 9: 1-11.

Agrobacterium transformation is widely used by those skilled in the art to transform dicotyledonous species. There are various approaches used for the routine production of stable, fertile transgenic plants in almost all economically relevant monocotyledonous plants [Toriyama, et al., (1988) Bio/Technology 6, 1072-1074; Zhang, et al., (1988) Plant Cell Rep. 7,379-384; Zhang, et al., (1988) Theor Appl Genet 76, 835-840; Shimamoto, et al., (1989) Nature 338, 274-276; Datta, et al., (1990) Bio/Technology 8, 736-740; Christou, et al., (1991) Bio/Technology 9, 957-962; Peng, et al., (1991) International Rice Research Institute, Manila, Philippines 563-574; Cao, et al., (1992) Plant Cell Rep. 11, 585-591; Li, et al., (1993) Plant Cell Rep. 12, 250-255; Rathore, et al., (1993) Plant Molecular Biology 21, 871-884; Fromm, et al., (1990) Bio/Technology 8, 833-839; Gordon-Kamm, et al, (1990) Plant Cell 2, 603-618; D'Halluin, et al., (1992) Plant Cell 4, 1495-1505; Walters, et al., (1992) Plant Molecular Biology 18, 189-200; Koziel, et al., (1993) Biotechnology 11, 194-200; Vasil, I. K. (1994) Plant Molecular Biology 25, 925-937; Weeks, et al., (1993) Plant Physiology 102, 1077-1084; Somers, et al., (1992) Bio/Technology 10, 1589-1594; and WO92/14828). In particular. Agrobacterium mediated transformation is now also emerging as a highly efficient alternative transformation method in monocotyledons (Hiei et al., (1994) The Plant Journal 6, 271-282).

The generation of fertile transgenic plants has been achieved in the cereals; rice, maize, wheat, oat, and barley [reviewed in Shimamoto, K. (1994) Current Opinion in Biotechnology 5, 158-162; Vasil, et al., (1992) Bio/Technology 10, 667-674; Vain et al., 1995, Biotechnology Advances 13 (4): 653-671; and Vasil, 1996, Nature Biotechnology 14 page 702].

Microprojectile bombardment, electroporation and direct DNA uptake are preferred where Agrobacterium is inefficient or ineffective. Alternatively, a combination of different techniques may be employed to enhance the efficiency of the transformation process, for example, bombardment with Agrobacterium coated microparticles (EP-A486234) or microprojectile bombardment to induce wounding followed by co-cultivation with Agrobacterium (EP-A486233).

Following transformation, a plant may be regenerated, for example, from single cells, callus tissue or leaf discs, as is standard in the art. Almost any plant can be entirely regenerated from cells, tissues and organs of the plant Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The present invention also extends to kits comprising the polynucleotide sequence of the present invention, suitably in combination with appropriate cloning vectors, to allow a gene of choice to be expressed under control of the polynucleotide sequence of the invention in in vivo or in vitro systems. Suitable kits include those in which a gene of interest can be easily cloned downstream of the sequence of the present invention, to produce a vector which may be used in plant cell transformation or in a cell free transcription tanslation assay for example. In the latter case, the kit may suitably comprise any necessary cell extracts and reagents.

The present invention also extends to a method for expressing a protein in a plant or plant expression system, comprising the steps of

1) linking the polynucleotide sequence of the present invention operably to a gene, open reading frame or polynucleotide sequence of interest;

2) expressing said gene, open reading frame or polynucleotide sequence in vivo or in vitro, such that the protein is produced; and

3) (optionally) purifying said protein.

The present invention also relates to methods for production of a transgenic plant, the method comprising the steps of:

1) linking the polynucleotide sequence of the present invention operably to a gene, open reading frame or polynucleotide sequence of interest;

2) recombining the linkage of step (1) into the genome of a suitable plant cell; and

3) growing a transgenic plant from the plant cell.

Methods to link polynucleotide sequences together are well known in the art, see for example, Sambrook et al., supra.

It is not necessary for the gene under translational control of the polynucleotide sequence of the present invention to be a plant gene. Genes or polynucleotide sequences from any organism may be used in conjunction with the polynucleotide sequence of the present invention, to produce protein in in vivo or in vitro systems. Accordingly, the present invention provides the possibility of easy purification of non-plant proteins in a plant or plant expression system, for example in the case where the non-plant protein has significantly different characteristics from other plant proteins.

The polynucleotide sequence of the present invention may be used to direct translation initiation of a single gene. The cap-independent nature of translation initiation may allow such a gene to be expressed even when a cell, for example, has been infected with a virus which disrupts the cap mechanism. Accordingly, the present invention may be used to permit expression of proteins to combat viral infection, for example, even when a host cell is not able to produce its own proteins, due to disruption of the cap system. Accordingly, the present invention also relates to a method for treatment of viral infection in plants, comprising insertion into a plant cell of a gene or genes under translation control of the polynucleotide sequence of the present invention, wherein the protein expressed by the gene or genes assists in combating viral infection.

The polynucleotide sequence of the present invention also allows bicistronic and multicistronic translation initiation. In general, the downstream cistron of a bicistronic mRNA is translated inefficiently because few ribosomes successfully scan from the termination codon of the first cistron to the second initiation codon and reinitiate. However, in the present invention, we have established that insertion of the 5′ UTR derived from RhPV between two reporter genes leads to efficient translation of the second ORF in a coupled rabbit reticulocyte (RRL) transcription and translation (TNT) reaction. Efficient IRES activity from the RHPV 5′ UTR in a wheat germ TNT system is also observed. Accordingly, the RhPV IRES may be used as an expression tool in plant systems to permit co-expression of two or more proteins from a single mRNA transcript. It will be understood that references herein to the RhPV 5′ UTR apply equally to other sequences of the present invention, unless otherwise apparent.

To achieve expression of a plurality of proteins or polypeptides from a single mRNA transcript, the RhPV IRES can be cloned between the genes of interest contained within an expression plasmid containing a promoter for transcription in plant cells, or may be used to link more than two genes together. For example, a simple construct may take the form of:

5′Promoter—Gene A—RhPV IRES—Gene B—3′ polyadenylation sequence

An example of a 5′ promoter that can be used is the CaMV 35S promoter.

This plasmid may then be introduced into plants by transformation. This may be achieved by a number of methods, including those used in assays for gene expression. Methods of transformation are well known to those skilled in the art. Examples of methods which can be used are microinjection, liposomes, Agrobacterium, PEG, gene gun etc. (c.f. Methods in Plant Molecular Biology—A Laboratory Course Manual, Cold Spring Harbor Laboratory Press, Eds Maliga et al., New York 1995).

Following transformation, transgenic plants may be created using methods readily familiar to those skilled in the art specific for different crops (c.f. Transgenic Plants; a production system for Industrial and Pharmaceutical proteins, Eds Owen and Pen, John Wiley & Sons Ltd., England, 1996).

For example, transgenic pea plants have been produced using the Agrobacterium method (Polowick et al., 2000, Plant Science, 153: 161-170). Transgenic wheat has been produced using methods outlined in Ingram et al., (2001, Acta Physiologiae Plantarum, 23: 221-239).

The present invention thus allows, for example: assembly of protein subunits into functional complexes; use of selectable markers in conjunction with a protein of interest; or expression of interacting proteins contributing to a single metabolic pathway in the same cell. An example of a such a pathway is an enzyme cascade.

For example, the above approach may be used to generate crops with particular traits, such as resistance to herbicides or insects, or to express other beneficial proteins. The cost and time required to insert multiple genes into a plant using this single transformation event approach is much less than traditional methods of crossing homozygous lines. The second, or subsequent, gene may also be a selectable marker gene, for example, an antibiotic resistance gene or green fluorescent protein (GFP).

In general, it will be appreciated that the sequences of the present invention can be used for the expression of any chosen gene in any appropriate system. More particularly, the sequences of the invention permit expression of genes in plant systems without the need for capping. Thus, associated genes may be expressed without some of the usual controls, or may be otherwise engineered for maximal expression, for example. More particularly, however, the sequences of the present invention are of use in polycistronic expression systems where it is not advantageous for expression of the linked genes to be attenuated. A sequence of the invention may then be positioned before one or more of the ORFs, as desired.

The present invention thus extends to the polynucleotide sequence of the present invention operably linked to a downstream cistron or gene in a multicistronic RNA. A single polynucleotide sequence of the present invention may be located between 2 ORF's or genes, to ensure both are translated. Alternatively, a copy of the polynucleotide sequence of the present invention may be inserted before each of a number of ORF's in a multicistronic RNA, to ensure that all are translated. The invention relates to all such combinations.

The present invention will now be illustrated with respect to the accompanying Figures and Examples, which serve to illustrate the present invention but are not binding thereon, wherein:

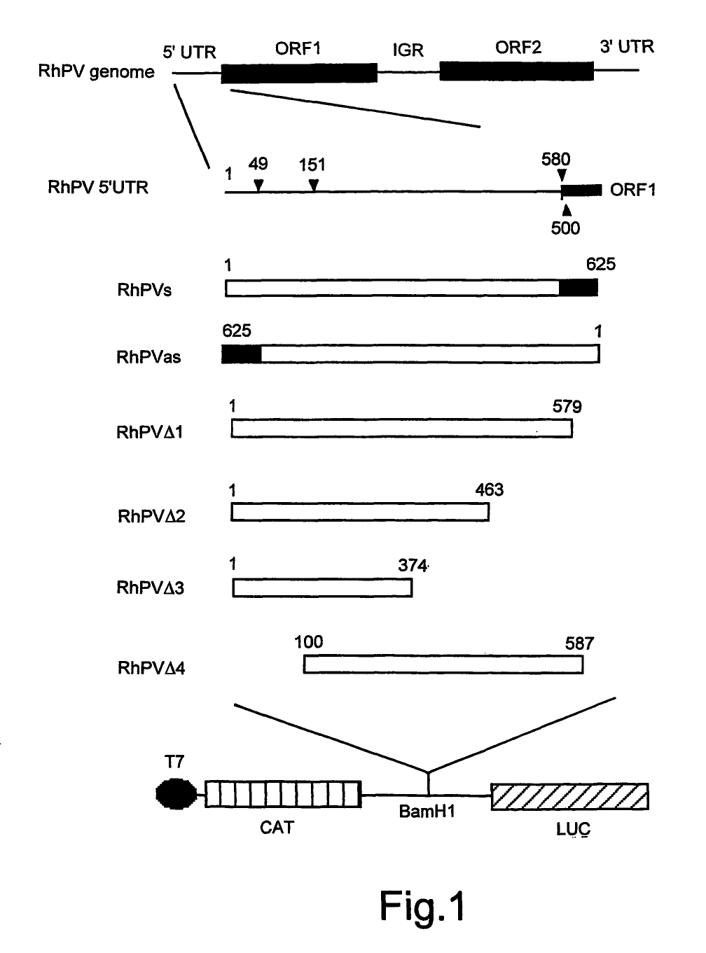

FIG. 1 illustrates the structure of the RHPV genome and plasmids used in this study;

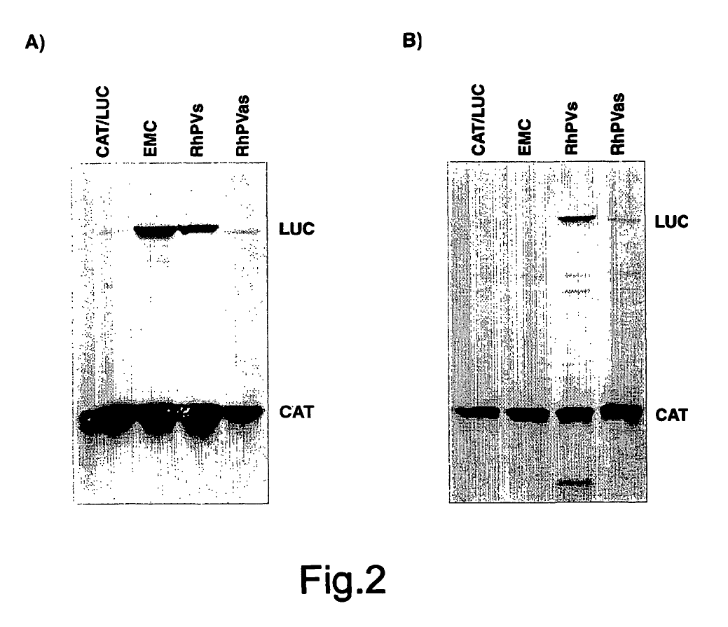

FIG. 2 illustrates that the RhPV 5′ UTR displays IRES activity in vitro;

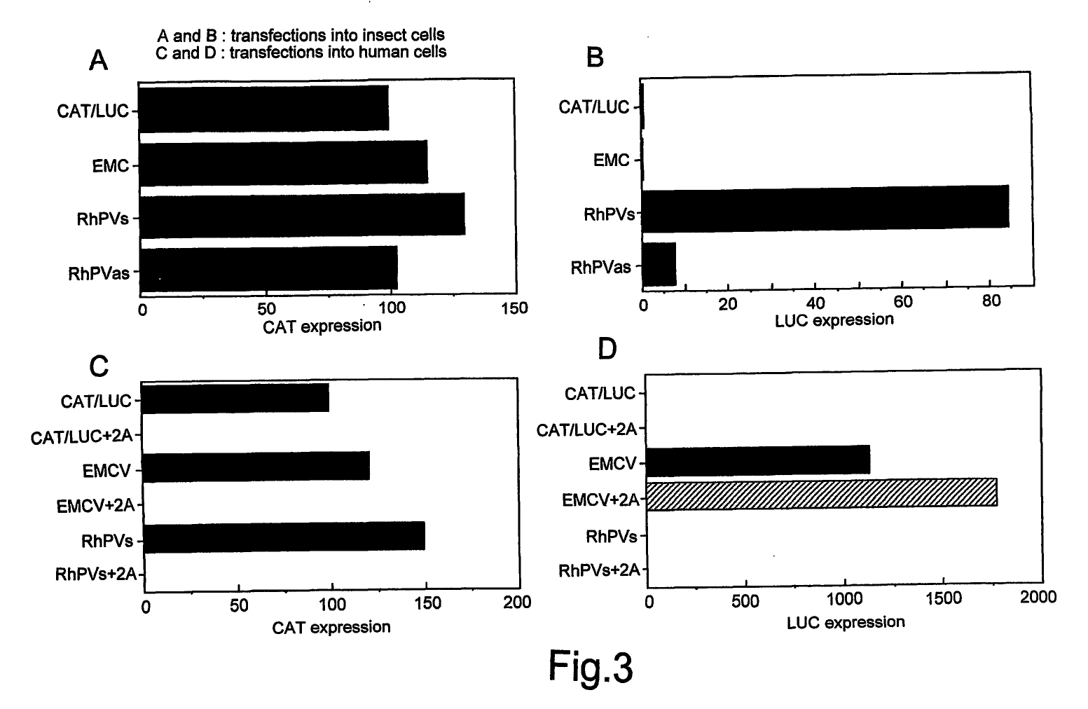

FIG. 3 illustrates that the RHPV 5′ UTR is inactive within human cells and that the RhPV 5′ UTR displays IRES activity in insect cells;

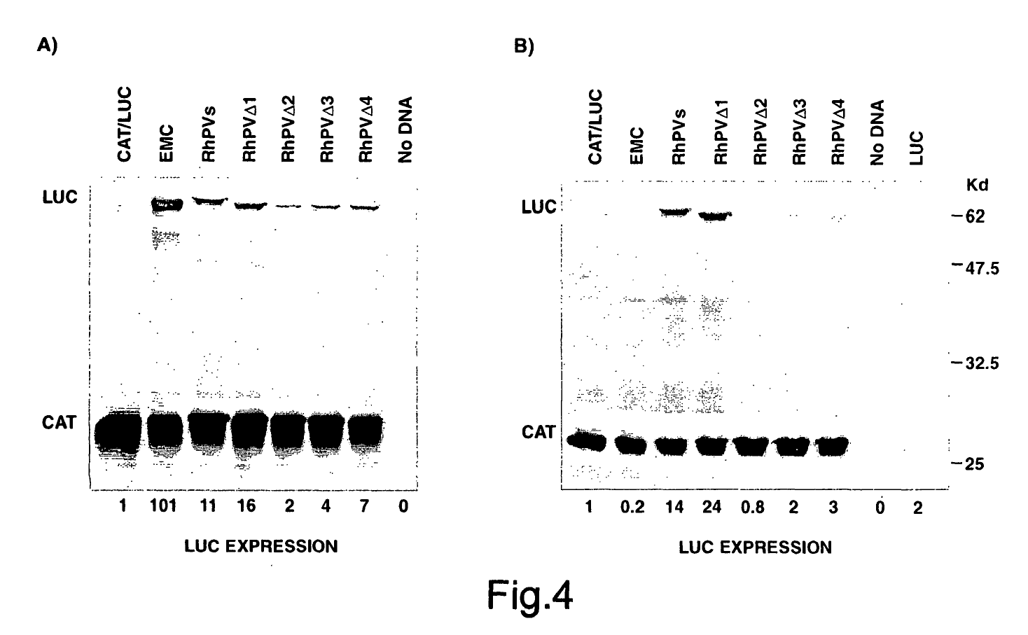

FIG. 4 illustrates delimitation studies of the RHPV 5′ UTR sequences required for IRES activity in vitro; and

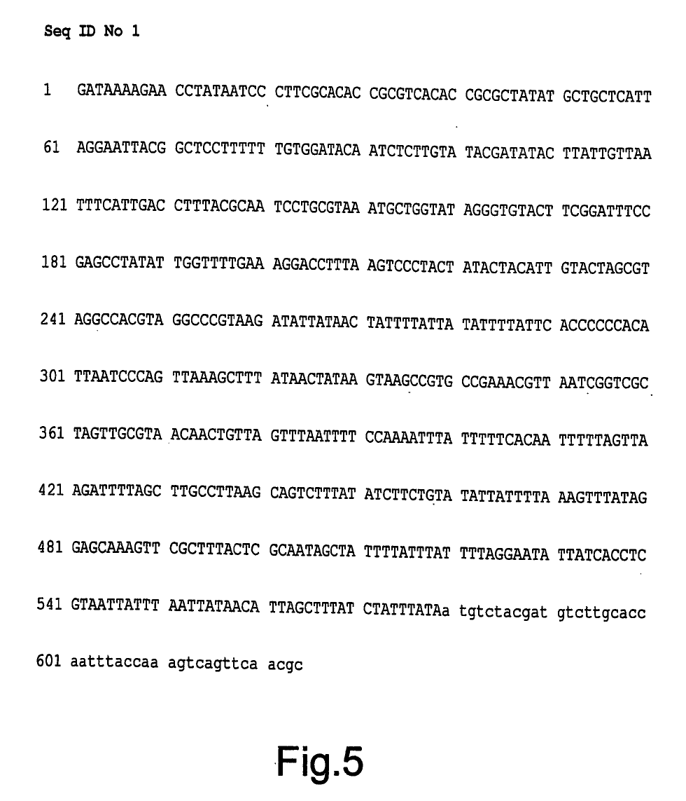

FIG. 5 illustrates the sequence of the RHPV 5′ UTR (in capital letters 5′ UTR only up to initiation codon, equivalent to sequence in RHPVΔ1 construct, with downstream RHPV ORF 1 DNA in normal type)

### EXAMPLE 1

### Analysis of the 5′ UTR of RhPV

A dicistronic reporter plasmid (pGEM-CAT/RhPVs/LUC, abbreviated RhPVs) was constructed in which the 5′ terminus of the RhPV genome (579 bases of UTR plus the first 15 codons of RhPV ORF1) was inserted between the coding sequences for chloramphenicol acetyl transferase (CAT) and luciferase (LUC) (FIG. 1). In this system IRES activity leads to the expression of the second gene (LUC) whereas cap-dependent translation is assayed as CAT expression. A second dicistronic construct was also prepared which contained the identical RhPV sequence, but inserted in the opposite (antisense) orientation (PGEM-CAT/RhPVas/LUC, abbreviated RhPVas).

Specifically, DNA preparations and manipulations were performed using standard methods as described in Sambrook et al., (supra) or by manufacturers of the reagents used. cDNA containing the 5UR and ORF1 sequence of RhPV was kindly donated by Dr L Domier (University of California). As the HCV IRES element has been shown to require some coding sequence for efficient function (Reynolds et al., 1995), sequence from the 5′ end of ORF1 was included in some of the constructs. For these, the 5′ UTR (nt 1-579) plus the first fifteen codons of the ORF1 sequence were amplified by PCR from RhPV cDNA using the forward primer RFOR1 (Table 1) and the reverse primer RREV625 (Table 1), each containing a BamHI site.

TABLE 1
Sequences of oligonucleotides
used to create RhPV 5′ UTR fragments

| Primer name | Primer sequence |
|---|---|
| RFORI | 5′ ATAGGATCCGATAAAAGAACCTATCACACCG |
| (SEQ ID NO. 2) | |
| RREV624 | 5′ TATGGATCCTGCGTTGAACTGACTTTGGT |
| (SEQ ID NO. 3) | |
| RREV579 | 5′ ACGGATCCTATAAATAGATAAAG |
| (SEQ ID NO. 4) | |
| RREV463 | 5′ ACGGATCCATATACAGAAGATAT |
| (SEQ ID NO. 5) | |
| RREV374 | 5′ ACGGATCCTTGTTACGCAACTAG |
| (SEQ ID NO. 6) | |
| RREV588 | 5′ ACGGATCCCGTAGACTATATAAA |
| (SEQ ID NO. 7) | |
| RFOR100 | 5′ ACGGATCCATACGATATACTTAT |
| (SEQ ID NO. 8) | |

BamHI sites highlighted in bold were added to enable cloning into the pGEM-CAT/LUC vector as described in the text

The PCR product was ligated into pGEMTeasy (Promega) and the structure of the created plasmids verified. The RhPV cDNA (624 nt) was then released by BamHI digestion and inserted in both orientations at the unique BamHI site of the dicistronic plasmid pGEM-CAT/LUC (van der Velden et al., Virology 214, 82-90 1995) between the two reporter genes (FIG. 1. The plasmid containing the RhPV 5′ UTR in the sense (genomic) orientation was designated pGEM-CAT/RhPVs/LUC (abbreviated RhPVs) and that containing this element in the antisense orientation was called pGEM-CAT/RhPVas/LUC (abbreviated RhPVas)). The structures of the plasmid constructs were verified by restriction enzyme digestion and sequencing. The dicistronic plasmnid pGEM-CAT/EMC/LUC (abbreviated EMC) containing the EMCV IRES element has been described previously (van der Velden et al, supra). The same 5′ portion of RhPV sequence was also inserted into a second dicistronic vector between the GUS and HOOK reporter genes (as described in Robertson, M. E. M., et al., (1999) RNA 5, 1167-1179) using the unique BamHI site of the vector pGUS/RXB/HOOK.

The activities of each plasmid were assessed in a coupled transcription-translation system (TNT Quick system, Promega) based on the rabbit reticulocyte lysate (RRL). Plasmid pGEM-CAT/EMC/LUC (abbreviated EMCV) containing the well-characterised EMCV IRES was used as a positive control, and plasmid pGEM-CAT/LUC which lacks any IRES sequences was used as a negative control.

Herein, where coupled transcription and translation reactions were used, the dicistronic plasmids (2 μg) were assayed in the rabbit reticulocyte lysate coupled transcription and translation system (TNT Quick system—Promega) or the wheat germ based coupled TNT system (Promega) essentially as described by the manufacturer. [³⁵S] methionine-labelled products were analysed on SDS-polyacrylamide gels and dried gels were exposed to Fuji X-ray film. Luciferase activity was measured using the luciferase assay system (Promega) and a Bio-orbit luminometer.

All plasmids induced efficient expression of CAT in RRL (FIG. 2A). Plasmids containing the RhPVs and EMCV constructs also produced high levels of LUC expression (FIG. 2A). The luciferase enzyme activity from the RhPVs plasmid was measured at about 30% of that observed with the EMCV IRES. However, the inclusion of 15 codons from RhPV ORF1 would donate an N-terminal extension to the luciferase protein and this may have affected luciferase activity of the protein. Little luciferase expression was detected with the RhPV 5′ UTR in the antisense orientation (RhPVas) or from the pGEM-CAT/LUC which contains no IRES element (FIG. 2A). The ability of the RhPVs sequence to promote internal initiation was also tested in a different context using a GUS/HOOK dicistronic construct as described previously (Robertson, M. E. M., et al., (1999) RNA 5, 1167-1179). In this case too, efficient expression of the second ORF was only achieved when the RHPV sequences were inserted between the GUS and HOOK ORF's in the sense orientation (data not shown). Thus we conclude that the RhPV 5′ terminus contains an IRES element that is active in the RRL system.

Specifically with respect to FIG. 1, various fragments of the 5′ end of the RhPV genome were amplified by PCR using primers containing BamHI sites, digested and inserted between the CAT and LUC ORF's (at the unique BamHI site) in the plasmid pGEM-CAT/LUC as described above. Nucleotide numbers corresponding to the fragments are shown. AUG codons within the RhPV 5′ UTR are indicated. The intergenic region (IGR) of RhPV is also indicated.

Specifically with respect to FIG. 2, plasmids encoding dicistronic mRNAs containing the viral sequences indicated were analysed in the in vitro transcription/translation systems as described above. Samples were analysed by SDS-PAGE and autoradiography. A) Translation in RRL. B) Translation in WGE. Relative luciferase activities were also measured and are expressed relative to the construct containing no IRES element (pGEM-CAT/LUC). Results shown are representative of three separate experiments.

### EXAMPLE 2

### Analysis of the RhPV IRES in the Wheat Germ Translation System

RhPV is believed to make use of plants only as passive vehicles for the transmission of infection to other aphids; but the virus genome does have some similarity to that of the comoviruses which do actively replicate in plants (Gildow, F. E., & D'Arcy, C. J. (1990) J. Invertebr. Pathol. 55, 245-257.1990). Thus it was possible that the reported lack of RhPV replication in plant cells might be due to a failure of IRES function in this environment. We therefore examined the ability of the RhPV IRES to direct translation initiation in the wheat germ translation system (WGE). Reporter plasmids containing the EMCV, RhPVs and RhPV as elements were analysed in a coupled T7 transcription/wheat germ translation system (Promega). As expected, efficient expression of CAT was observed from all plasmids. The EMCV IRES was totally inactive in this plant system and decreased LUC expression compared to the CAT/LUC control lacking any IRES element. However, luciferase was very efficiently expressed from the RhPVs construct (17 fold above the CAT/LUC control) and once again this expression was abrogated if the IRES sequences were present in the antisense form (FIG. 2B). The CrPV IGR has also recently been reported to function in the WGE system (Wilson, J. E., et al., (2000) Mol. Cell Biol. 20, 4990-4999). However, our data contrasts with the inactivity of the CrPV 5′ UTR in the WGE system reported by these authors.

### EXAMPLE 3

### Analysis of the function of RHPV IRES in Human and Insect Cells

RhPV infects only a narrow range of aphid species; host cell-dependent restriction of IRES function could be a possible contributor to the determination of host range and we therefore examined the ability of the RhPV IRES to function within insect cells. The plasmid constructs described above were introduced into a non-aphid cell line Sf21, derived from Spodoptera frugiperda, and human TK-143 cells.

Transfections were carried out as described previously (Roberts, L. O. et al., (1998) RNA 4, 520-529.). In brief, human TK-143 cells were grown in 35 mm dishes and infected with the recombinant vaccinia virus vTF7-3, which expresses T7 RNA polymerase (Fuerst, et al., (1986) Proc. Natl. Acad Sci. U.S.A. 83, 8122-8126). At 1 h post-infection dicistronic plasmids (2 μg) were transfected into the cells using with Lipofectin (8 μg; Life Technologies) and Optimem (Gibco). Plasmids were transfected either alone or in combination with pAα802 (0.5 μg) in order to express poliovirus protein 2A. The plasmid pAΔ802 encoding poliovirus protease 2A (PV 2A) was supplied by Dr A Kaminski (University of Cambridge) and has been described previously (Kaminski, A., et al., (1990) EMBO J. 9, 3753-3759). After 20 h, cell extracts were prepared in Promega lysis buffer (400 μl). Insect cells (Sf21) were transfected in a similar manner except that expression of T7 RNA polymerase was induced by prior infection for 4 h with the recombinant baculovirus AcMNPV/T7N (van Poelwijk, Broer, R., et al.,(1995) Bio/Tech. 13,261-264), kindly provided by Dr J Vlak, Agricultural University, Wageningen, Netherlands. Plasmids were transfected into the insect cells using Lipofectin (10 μl) and TC100 medium (minus serum). After 50 h, cell extracts were prepared in Promega lysis buffer (200 μl) as above. All samples were assayed for CAT expression using the CAT-ELISA kit (Boehringer Mannheim) and for LUC expression using the Promega luciferase assay kit as above.

T7 RNA polymerase expression was induced in both cell types by prior infection with a recombinant virus expressing this protein; recombinant vaccinia virus (Fuerst et al., supra) was used for TK-143 cells and recombinant baculovirus (van Poelwijk et al., 1995, supra) for the Sf21 cells. Cell extracts were made in lysis buffer and assayed for CAT and LUC expression. In vaccinia virus vTF7-3-infected human TK- cells, efficient CAT expression was obtained from each plasmid (FIG. 3C). However, surprisingly, only the EMCV IRES induced efficient luciferase expression in these cells, RhPVs and RhPVas failed to induce significant levels of luciferase (FIG. 3D). Thus, although RhPVs is functional in rabbit reticulocyte lysates it was apparently non-functional in this human cell line. Co-expression of poliovirus protease 2A (from pAΔ802) has been shown to stimulate low IRES activity within cells (Roberts, L. O. et al., (1998) RNA 4, 520-529). Co-transfections were carried out with the IRES-containing plasmids and pAΔ802. Although expression of PV 2A protease did significantly inhibit the cap-dependent synthesis of CAT as predicted (FIG. 3, panel C), it had no stimulatory effect on the activity of the RHPV IRES (FIG. 3, panel D).

Strikingly different results were obtained using the insect cells. As expected, all plasmids directed CAT expression (FIG. 3 panel A). The EMCV IRES was found to be very inefficient in these cells in agreement with a recent report (Finkelstein, Y. et al., (1999) J. Biotech. 75, 33-44). In contrast, the RHPV 5′ UTR directed luciferase expression to levels 70 fold above the background expression obtained from the negative control plasmid (pGEM-CAT/LUC) and over 100 fold higher than the EMCV IRES (FIG. 3 panel B). Thus the RhPVs construct displayed IRES activity within insect cells but not within human cells, whereas the EMCV IRES behaved in the opposite manner functioning efficiently in human, but not insect cells.

Specifically with respect to FIG. 3 panels C and D, the dicistronic plasmids were transfected into vTF7-3-infected TK-143 cells alone, or with pAΔ802 (which expresses PV 2A) as described above. Cell extracts were prepared 20 h later and samples analysed for LUC activity (C) using a luciferase assay system (Promega) and CAT expression (D) using the CAT ELISA (Boehringer Mannheim). LUC activity is plotted in arbitrary units and CAT expression relative to the pGEM-CAT/LUC plasmid. The IRES-containing plasmids are referred to by the name of the IRES insert. Results are representative of four separate experiments.

Specifically with respect to FIG. 3 panels A and B, the dicistronic plasmids were transfected into AcMNPV/T7-infected Sf21 cells as described above. Cell extracts were prepared 50 h later and samples analysed for LUC activity (A) using a luciferase assay system (Promega) and CAT expression (B) using the CAT ELISA (Boehringer Mannheim). LUC activity is plotted in arbitrary units and CAT expression relative to the pGEM-CAT/LUC plasmid. The IRES-containing plasmids are referred to by the name of the IRES insert. Results are representative of four separate experiments.

### EXAMPLE 4

### Mapping the 5′ and 3′ Boundaries of the RhPV IRES

Attempts to delineate the 5′ and 3′ boundaries of the RHPV 5′ UTR IRES were performed by construction of deleted versions in which sequences were removed from either end of the insert contained in the RhPVs construct. Truncated versions of the RhPVs were generated by PCR and cloned into the dicistronic CAT/LUC vector as described above (FIG. 1).

Specifically, three truncated versions of the RhPVs plasmid were constructed containing 3′ end deletions: RHPVΔ1 was created by PCR amplification using primers RFOR1 and RREV579. This construct contains the probable complete 5′ UTR (nt 1-579) but terminates immediately upstream of the predicted initiation codon at nt 580; RhPVΔ2 contains nt 1-463 and was created using primers RFOR1 and RREV463; RhPVΔ3 contains nt 1-374 and was amplified using primers RFOR1 and RREV374. Finally, a single 5′ end deletion was constructed containing nt 100-588 (RhPVΔ4) using primers RFOR100 and RREV588. Primer sequences are given in Table I and the 5′UTR fragments created are illustrated in FIG. 1 and were also inserted in the CAT/LUC vector as described above.

Each plasmid was tested in the rabbit reticulocyte and wheat germ coupled transcription/translation systems. Deletion of the coding sequence from the RhPVs construct (RhPVΔ1) had no negative effect on the amounts of LUC expressed from the construct in the RRL. Note the faster migration of the LUC protein produced from this construct compared to the fusion protein generated by the inclusion of 15 amino acids of viral coding sequence in the RhPVs construct (FIG. 4A). Quantitation of luciferase activity showed that enzyme activity was greater than that observed from the construct containing viral coding sequence. Similarly, activity of the RhPV IRES measured by protein synthesis in the wheat germ system, was not inhibited by the deletion of these codons. An increase in LUC activity was observed by the removal of the viral coding sequence from the constructs (1.5 fold increase; FIG. 4B). Thus, in common with picornavirus IRES elements, but unlike the HCV IRES element, the RhPV IRES does not extend into the virus coding sequence. Deletion of the 3′ end of the RhPV 5′ UTR (removal of nt 464-579) formed construct RhPVΔ2. This deletion significantly affected the ability of the 5′ UTR to direct internal initiation in both translation systems (FIG. 4A and B), and luciferase expression fell to about 13% of that directed by the full-length 5′ UTR in RhPVΔ1. Intriguingly, removal of a further 89 nucleotides from the 3′ end (construct RhPVΔ3; removal of nt 375-579) partially restored IRES activity (25% versus RhPVΔ1). Deletion of the first 99 bases from the 5′ end of RhPVs gave rise to construct RhPVΔ4. This deletion had a smaller effect on luciferase activity (44% loss versus the RhPVΔ1 construct) in both coupled transcription/translation systems indicating that these 5′ sequences may be less critical for IRES function (FIG. 4A and B).

Specifically with respect to FIG. 4, dicistronic plasmids containing the RhPV 5′ UtR and truncated versions of this sequence were analysed in the RRL (A) and WGE (B) coupled transcription/translation systems as described in Materials and Methods. Samples were analysed by SDS-PAGE and autoradiography. LUC activity was also measured using the Promega luciferase assay kit and values are plotted relative to the activity obtained with no IRES element (plasmid pGEM-CAT/LUC). Results are representative of two separate experiments.

1. A polynucleotide sequence capable of directing translation initiation in in vitro plant expression systems in a cap-independent manner and which directs translation initiation from an AUG or ATG codon, characterised in that the polynucleotide sequence comprises the 5′ UTR of RhPV, which has the sequence of SEQ ID NO. 1, or a variant thereof, the variant being capable of hybridising, when there is about 80% identity or more, with the antisense polynucleotide of SEQ ID NO. 1, under stringent conditions comprising 750 mM NaCl and 75 mM trisodium citrate, or less.

2. A sequence according to claim 1, which is ss or ds DNA.

3. A sequence according to claim 1 or 2, which comprises an IRES element.

4. A vector comprising at least one sequence according to any preceding claim.

5. A vector according to claim 4, wherein the sequence is operably linked to one or more genes or open reading frames.

6. A vector according to claim 5, wherein the first or only gene or open reading frame encodes a naturally occurring polypeptide having a 5′ methionine.

7. A vector according to any of claims 4 to 6, wherein the expression of a selectable marker is operably linked with the sequence.

8. A vector according to claim 7, wherein at least a second gene or open reading frame is operably linked with the sequence.

9. An expression system containing a vector according to any of claims 4 to 8.

10. A system according to claim 1, which is a plant cell or plant tissue.

11. A plant containing a vector according to any of claims 4 to 8.

12. A method for expressing a protein in a plant or plant expression system, comprising the steps of
1) linking a polynucleotide sequence according to any of claims 1 to 3 operably to a gene, open reading frame or polynucleotide sequence of interest;
2) expressing said gene, open reading frame or polynucleotide sequence in vivo or in vitro, such that the protein is produced; and
3) optionally purifying said protein.

13. A method for the production of a transgenic plant, comprising the steps of:
1) operably linking a polynucleotide sequence according to any of claims 1 to 3 to a gene, open reading frame or polynucleotide sequence of interest;
2) recombining the linkage of step (1) into the genome of a suitable plant cell; and
3) culturing said plant cell to produce said transgenic plant.

14. A method for treatment of viral infection in plants, comprising insertion into a plant cell of a gene or genes under translation control of a polynucleotide sequence according to any one of claims 1 to 3, wherein the protein expressed by the gene or genes assists in combating viral infection.

15. A vector comprising a polynucleotide sequence according to any of claims 1 to 3 operably linked to a gene, open reading frame or polynucleotide sequence of interest, for use in the treatment of a viral infection in plants.


SEQUENCE LISTING (8 sequences)

SEQ ID NO: 1 - DNA, 624 bp, Rhopalosiphium padi virus
  5′UTR (1)..(579)
gataaaagaacctataatcccttcgcacaccgcgtcacaccgcgctatatgctgctcattaggaattacggctccttttttgtggatacaatctcttgtatacgatatacttattgttaatttcattgacctttacgcaatcctgcgtaaatgctggtatagggtgtacttcggatttccgagcctatattggttttgaaaggacctttaagtccctactatactacattgtactagcgtaggccacgtaggcccgtaagatattataactattttattatattttattcaccccccacattaatcccagttaaagctttataactataagtaagccgtgccgaaacgttaatcggtcgctagttgcgtaacaactgttagtttaattttccaaaatttatttttcacaatttttagttaagattttagcttgccttaagcagtctttatatcttctgtatattattttaaagtttataggagcaaagttcgctttactcgcaatagctattttatttattttaggaatattatcacctcgtaattatttaattataacattagctttatctatttataatgtctacgatgtcttgcaccaatttaccaaagtcagttcaacgc

SEQ ID NO: 2 - DNA, 31 bp, Artificial Sequence
  RFOR1 PCR Primer
ataggatccgataaaagaacctatcacaccg

SEQ ID NO: 3 - DNA, 29 bp, Artificial Sequence
  RREV624 PCR Primer
tatggatcctgcgttgaactgactttggt

SEQ ID NO: 4 - DNA, 23 bp, Artificial Sequence
  RREV579 PCR Primer
acggatcctataaatagataaag

SEQ ID NO: 5 - DNA, 23 bp, Artificial Sequence
  RREV463 PCR Primer
acggatccatatacagaagatat

SEQ ID NO: 6 - DNA, 23 bp, Artificial Sequence
  RREV374 PCR Primer
acggatccttgttacgcaactag

SEQ ID NO: 7 - DNA, 23 bp, Artificial Sequence
  RREV588 PCR Primer
acggatcccgtagactatataaa

SEQ ID NO: 8 - DNA, 23 bp, Artificial Sequence
  RFOR100 PCR Primer
acggatccatacgatatacttat